(19) United States
(12) Patent Application Publication
(10) Pub. No.: US 2004/0220599 A1
(43) Pub. Date: Nov. 4, 2004

(54) Device for separating the epithelium layer from the surface of the cornea of an eye

(75) Inventors: Ioannis Pallikaris, Gazi of Heralklion (GR); Harilaos S. Ginis, Heraklion (GR)

Correspondence Address: BRINKS HOFER GILSON & LIONE, P.O. BOX 10395, CHICAGO, IL, 60610, US

(73) Assignee: FOS HOLDING S.A.

(21) Appl. No.: 10/787,026

(22) Filed: Feb. 25, 2004

Related U.S. Application Data
(63) Continuation of application No. 10/098,167, filed on Mar. 12, 2002.
(63) Continuation-in-part of application No. 09/911,356, filed on Jul. 23, 2001.

(51) Int. Cl. A61F 9/00

(52) U.S. Cl. 606/166

(57) ABSTRACT

A device is disclosed for separating the epithelial layer of a cornea from the eye. The device includes a separator having an edge to remove the epithelial layer as the separator moves across the eye. The edge includes a thickness thicker than the thickness of at least one epithelial cell and less thick than the thickness of the epithelial layer. Separation can be performed mechanically, without the use of chemicals, so that the shape and integrity of the separated epithelial layer is preserved. The device can also be used with a polymer film that adheres to the epithelial layer to help preserve an integrity of the epithelial layer.

### REFERENCE TO EARLIER FILED APPLICATION

The present application claims the benefit as a Continuation-in-Part of U.S. patent application Ser. No. 09/911,356 filed Jul. 23, 2001, which is incorporated by reference herein.

### BACKGROUND

LASIK (Laser-Assisted In Situ Keratomileusis) is a surgical procedure intended to reduce a person's dependency on glasses or contact lenses. LASIK permanently changes the shape of the cornea, the clear covering of the front of the eye, using an excimer laser. A device, called a microkeratome, is used to cut a flap in the cornea. A hinge is left at one end of this flap. The flap is folded back revealing the stroma, the middle section of the cornea. Pulses from a computer-controlled laser vaporize a portion of the stroma and the flap is replaced. It is important that the knife used during the LASIK procedure is sharp, otherwise the quality of the procedure and the healing time are poor. Additionally the knife has to be sharp in order to produce consistent and reproducible flaps. There are some complications related to the use of microkeratomes. Common complications include the creation of an irregular flap, for example, a half flap, a buttonhole, or a total cup. These complications represent irregular incisions of the cornea, a situation that can permanently degrade visual performance.

Alternatively, PRK (Photo-Refractive Keratectomy) which is a technique developed earlier than LASIK may be used to correct the curvature of the cornea. In PRK a physician scrapes away the superficial layer, e.g., the epithelium, of the cornea. After the superficial layer is removed, laser treatment is applied on to the exposed surface of the cornea. A drawback of PRK, however, is that the healing period for the eye typically lasts for a week, much longer than the healing period of LASIK. Also, the patient experiences some pain during healing. Typically in PRK a disposable contact lens is used to cover the treated area of the cornea and help reduce postoperative pain.

In another technique, LASEK (Laser Epithelial Keratomileusis) the epithelial layer is separated from the surface of the cornea in a manner that the separated epithelial layer can be preserved. First, the epithelium is treated with and alcohol solution to partially devitalize it. Once the exact surface area of treatment is determined, a few drops of a weak alcohol solution is applied to the surface of the cornea and allowed to stay in contact with the epithelium for a few seconds. This weak alcohol solution is then rinsed off the surface of the eye. The function of the weak alcohol solution is to loosen the epithelial layer (50 microns) and to allow it to be peeled back in a sheet of epithelial cells, thereby exposing the underlying cornea. This is not to be confused with LASIK, which actually uses a microkeratome instrument to create a flap of both epithelium and the front part of the stromal tissue measuring anywhere between 130 to 180 microns.

In LASEK, the epithelium-only layer is laid back in a similar fashion to LASIK, but consists of only epithelium, not corneal stroma. Once the epithelial cells have been laid out of the way, the laser is applied to the surface of the cornea in the exact same fashion as in PRK. Once the laser treatment has been completed, the epithelial layer is laid back into place and a soft contact lens is placed over the eye as in PRK. The epithelial cells, which were partly devitalized by the weak alcohol solution, are laid over the treatment area and may serve as a facilitator of new epithelium healing underneath. The alcohol-devitalized epithelium falls off the eye, similar to a scab, in 5-10 days. These devitalized epithelial cells do not become the new surface of the eye, but simply serve as a protective agent in addition to the contact lens to facilitate comfort and healing of the new underlying epithelium. Alcohol treatment of the epithelium results in a severe amount of epithelial cell loss, a fact that may render the epithelial disk not usable, due to the reduced durability and adhesion on to the cornea.

Thus, there is a need for an automated corneal epithelium separator that addresses the above problems by separating the epithelial layer as a whole in a mechanical way, not chemical.

### BRIEF SUMMARY

To help correct an imperfect vision of a patient's eye, an automated mechanical device separates the epithelial layer from the cornea of a patient's eye from the cornea. After the epithelial layer is separated from the cornea, a laser is used to help correct imperfections in the cornea. Thereafter, the epithelial layer is placed back on the cornea to reduce the visual rehabilitation period and reduce postoperative pain.

In one aspect, the device includes a separator such as a plate, wire or dull blade. The device can preserve a separated epithelial layer as a disk without rupturing the disk and without substantial epithelial cell loss. The epithelial layer is separated from the cornea without cutting the cornea.

The device includes a separator having an edge to remove the epithelial layer as the separator moves across the eye. The edge includes a thickness thicker than the thickness of at least one epithelial cell and less thick than the thickness of the epithelial layer.

### BRIEF DESCRIPTION OF THE DRAWINGS

FIG. 1 is a diagram showing a side view of an eye and a epithelial separator with a separator located in a first position according to the preferred embodiments.

FIG. 2 is a diagram showing a top view of the eye and the separator located in a first position according to the preferred embodiments.

FIG. 3 is a diagram showing a side view of the eye and the separator located in a second position according to the preferred embodiments.

FIG. 4 is a diagram showing a top view of the eye and the separator located in a second position according to the preferred embodiments.

FIG. 5 is a diagram showing a side view of the eye and the separator located in a third position according to the preferred embodiments.

FIG. 6 is a diagram showing a top view of the eye and the separator located in a third position according to the preferred embodiments.

FIG. 7 is a diagram showing a side view of the eye and the separator located in a fourth position according to the preferred embodiments.

FIG. 8 is a diagram showing a top view of the eye and the separator located in a fourth position according to the preferred embodiments.

FIG. 9 is a diagram showing a top view of the eye and the separator located in a fifth position according to the preferred embodiments, the separator is retracted after epithelial separation.

FIG. 10 is a diagram showing a top view of the eye with the separator removed.

FIG. 11 is a diagram showing a top view of the eye after ablations is performed with a laser.

FIG. 12 is a diagram showing a top view of the eye with the epithelium replaced on the eye.

FIG. 13. is a diagram showing a top view of the eye with the epithelium smoothly stretched into place.

FIG. 14 is a diagram showing a side view of the eye and the epithelial separator device including a rotating drum.

FIG. 15 is a diagram showing a front view of the eye and the epithelial separator device including the rotating drum.

FIG. 16 is a diagram showing a top view of the eye and the epithelial separator device including the rotating drum.

FIG. 17 is a diagram showing a drum according to one embodiment.

FIG. 18 is a diagram showing a drum according to another embodiment.

FIG. 19 is a diagram representing a side view of a separator removing the epithelial layer from the Basal membrane of the eye.

FIG. 20 is a diagram showing a perspective view of a known blade.

FIG. 21 is a diagram showing a side view of a separator's leading edge according to an embodiment.

FIG. 22 is a diagram showing a side view of a separator's leading edge according to another embodiment.

FIG. 23 is a diagram showing a side view of a separator's leading edge according to yet another embodiment.

FIG. 24 is a diagram showing a perspective view of a wire that could be used as a separator according to a preferred embodiment.

FIG. 25 shows a perspective view of an exemplary machine that is used to condition a separator according to one embodiment.

FIG. 26 shows a front view of the machine of FIG. 25 including the separator.

FIG. 27 shows a side view of one embodiment of a device for separating and preserving an epithelial layer.

FIG. 28 shows a top view of the device of FIG. 27.

### DETAILED DESCRIPTION

To help correct an imperfect vision of a patient's eye, an automated mechanical device separates the epithelial layer from the cornea of a patient's eye from the cornea. A separator, such as a plate, wire or dull blade is used to separate the epithelial layer of the cornea from the basal membrane. In this way, the automated mechanical device can preserve the separated epithelial layer as a disk without rupturing the disk and without substantial epithelial cell loss, less than 5-10% loss, to ensure viability and stability of the epithelial disk after replacement on the surface of the cornea. After the epithelial layer is separated from the cornea, a laser is used to help correct imperfections in the cornea. Thereafter, the epithelial layer is placed back on the cornea to aid in the healing process of the eye.

FIG. 1 is a diagram showing a side view of an eye 10 of a patient and a epithelial separator device 12. The epithelial separator device 12 includes a separator 14, shown here in a first position located away from the eye 10. The separator 14 includes a device that can scrape the epithelium from the cornea such as a plate, a wire or a knife with a dull edge. The separator 14 removes an epithelium layer 16 located above a corneal surface 18 of the eye 10. The separator 14 is not sharp enough to excise corneal tissue during operation of the epithelial separator device 12.

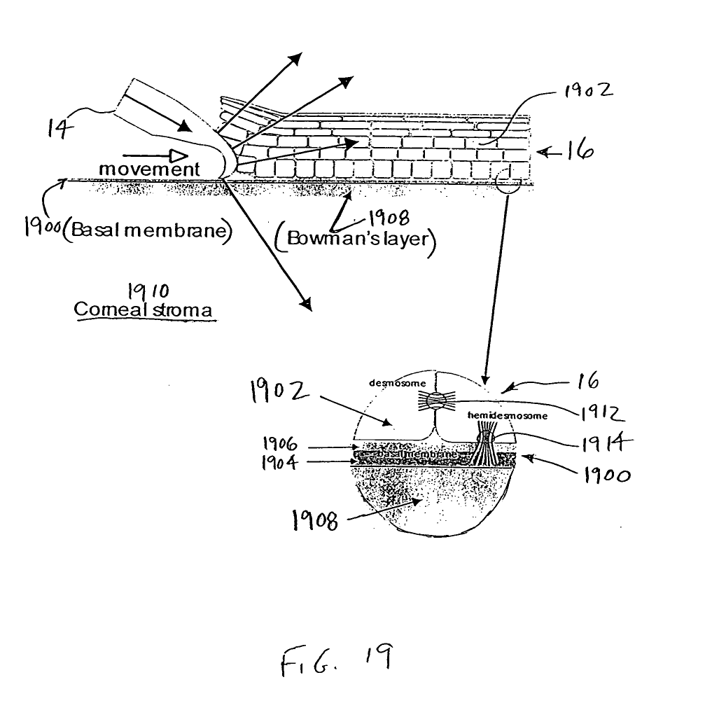

Referring also to FIG. 2, the epithelial separator device 12 includes a ring 20 that sits on the eye 10 with its plane parallel to a limbus of the eye. The ring 20 includes an internal diameter 22 ranging from about 10 to about 12 mm and external diameter 24 from about 13 to about 16 mm and including a groove 26 (best seen in FIG. 15). The groove 26 is dimensioned wider than the internal diameter 22. A separator support 28 fits in the groove 26 to carry the separator 14 on a determined travel.

An oscillation device 30 provides motion and vibration to the separator 14. The oscillation device 30 can oscillate the separator 14 either transversely or longitudinally with frequency ranging from about 10 Hz to about 10 KHz. Electromagnetic or piezoelectric forces on the separator 14 can provide the oscillation, or external rotating or vibrating wires can provide the oscillation. To maintain the ring 20 on the eye 10, for example during oscillation, the ring 20 can include a circumferential groove 32 positioned on a side of the eye 10. Suction can be applied to the circumferential groove 32 to ensure stable mounting of the ring 20 to the eye 10.

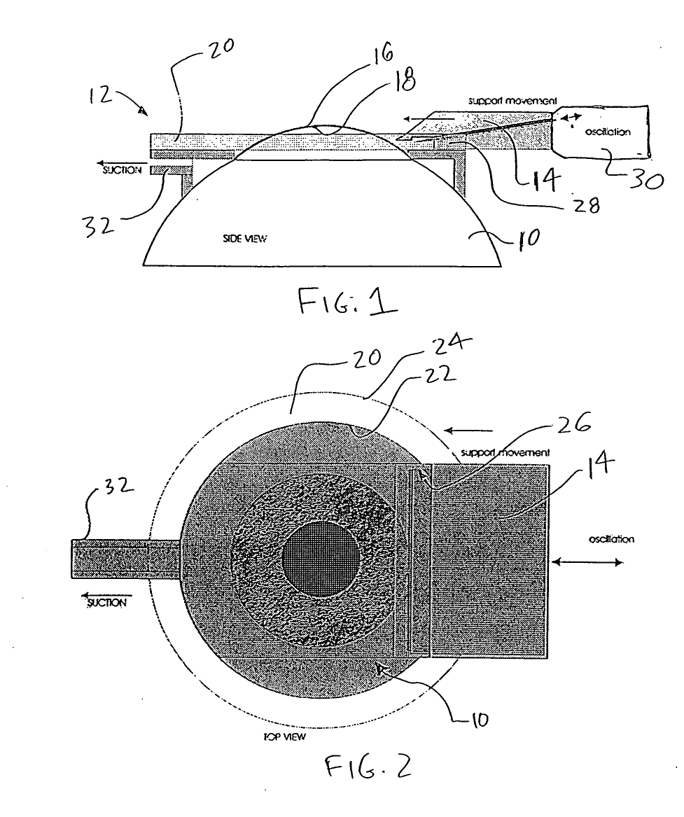

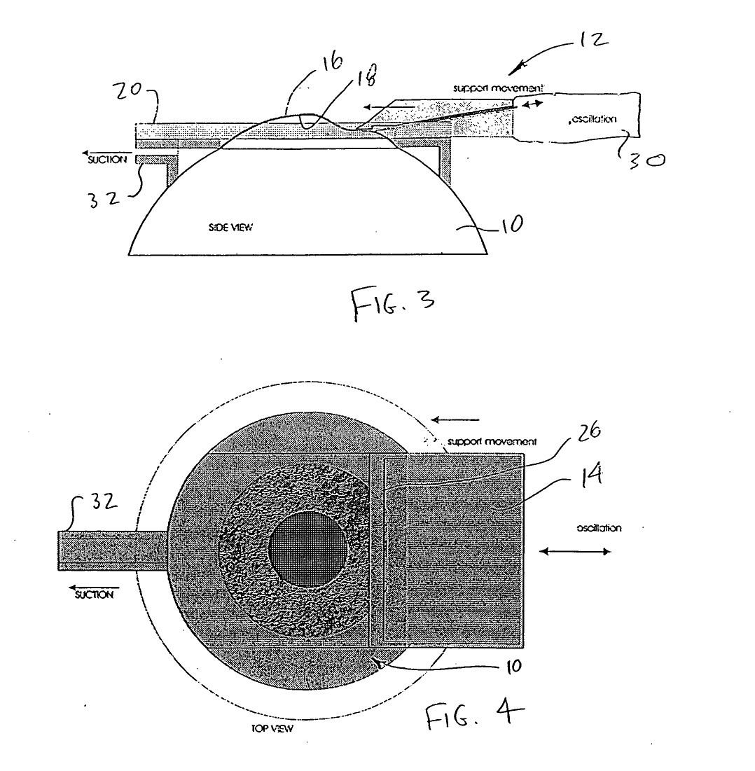

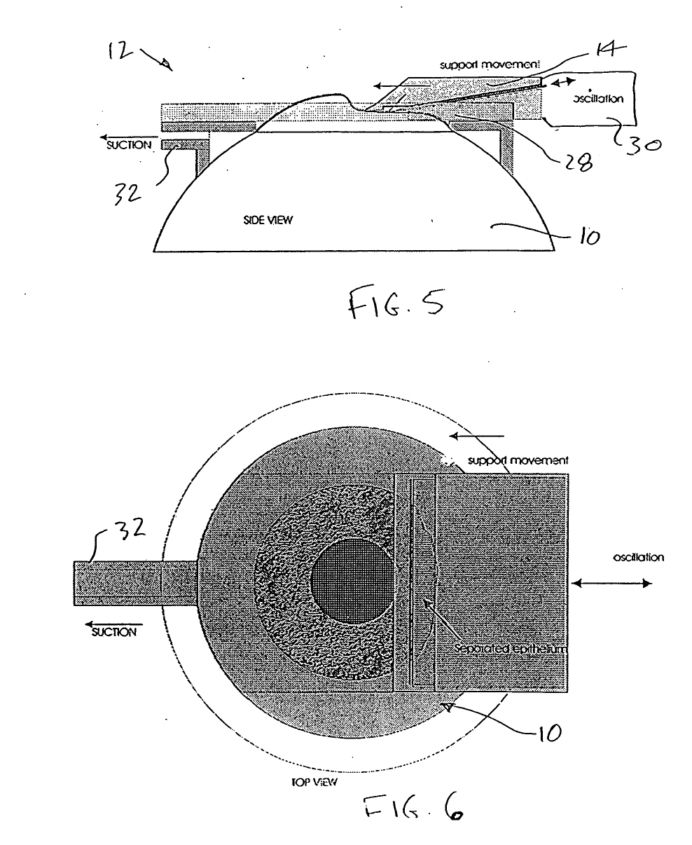

FIGS. 3 and 4 are diagrams showing a side and a top view, respectively, of the eye 10 and the separator 14 located in a second position with respect to the eye. As the separator 14 travels to contact the eye 10, the corneal surface 18 is flattened. To accommodate the travel of the separator 14, the separator support 28 freely slides in the groove 26, for example, when driven by the oscillation device 30.

FIGS. 5 and 6 are diagrams showing a side and a top view of the eye 10 and the separator 14 located in a third position. As the separator 14 travels along the cornea 10, the epithelium layer 16 is separated from the cornea. The separator 14 separates the epithelium layer 16 without cutting the cornea 18.

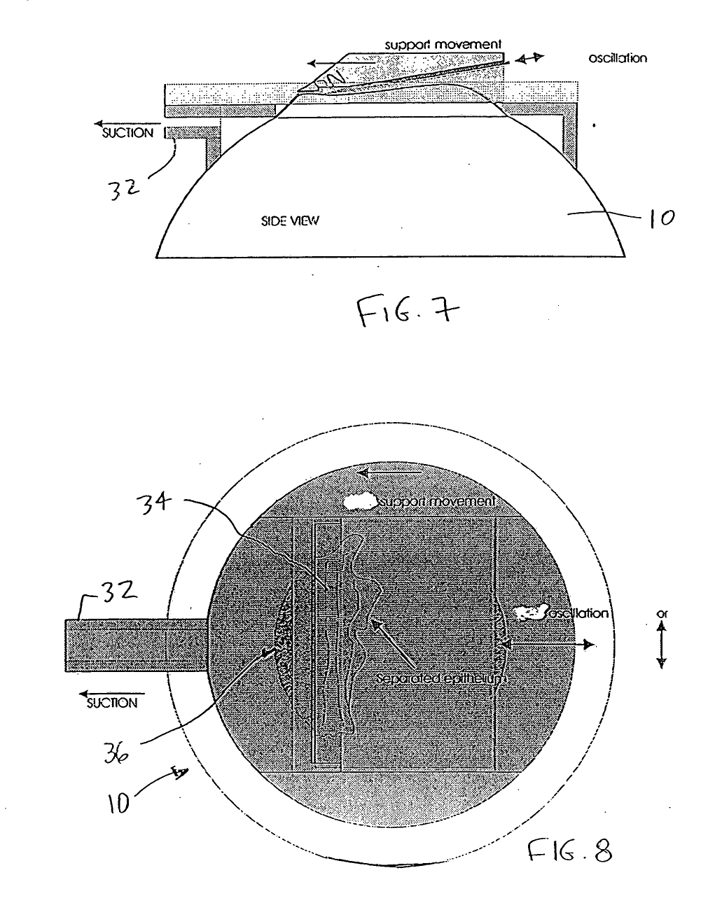

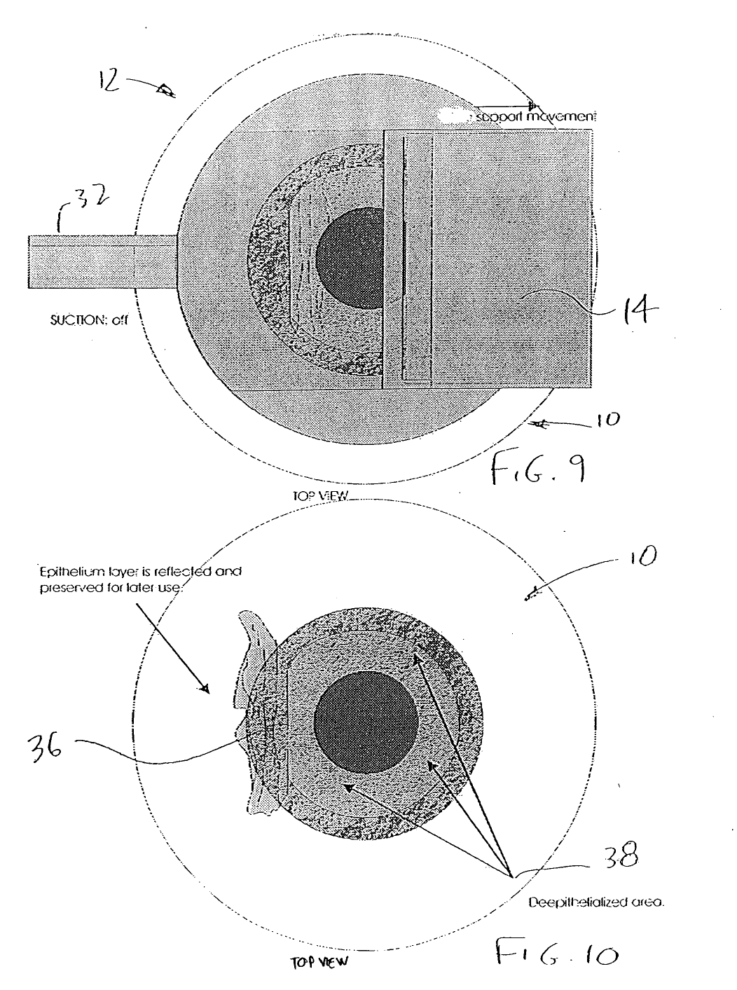

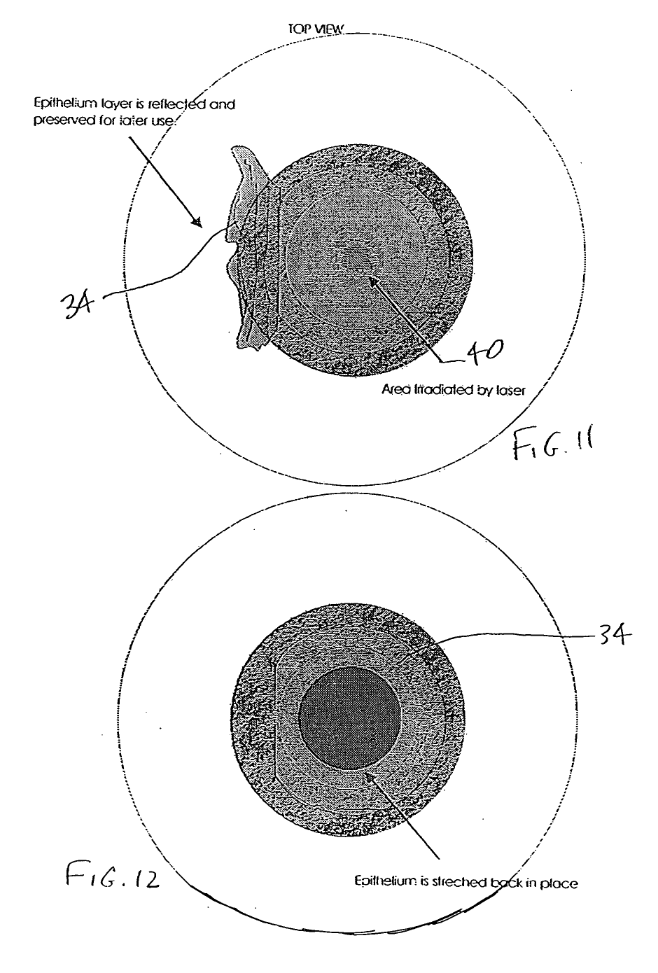

FIGS. 7 and 8 are diagrams showing a side and a top view of the eye 10 and the separator 14 located in a fourth position. In one embodiment, the travel of the separator 14 is controlled to produce an epithelial disk 34 hinged at an edge 36 of the epithelial disk 34. In another embodiment the epithelial disk 34 is completely detached for the corneal surface 18, for example, as described below.

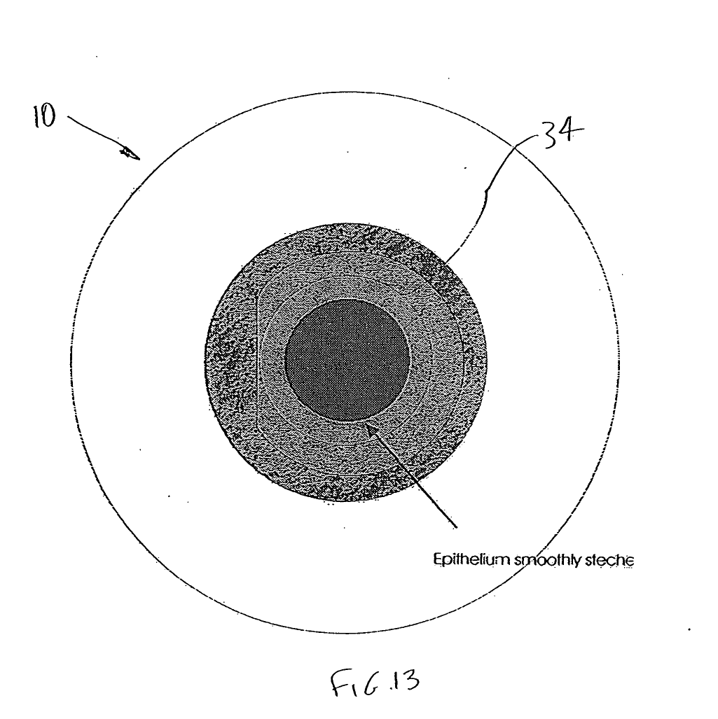

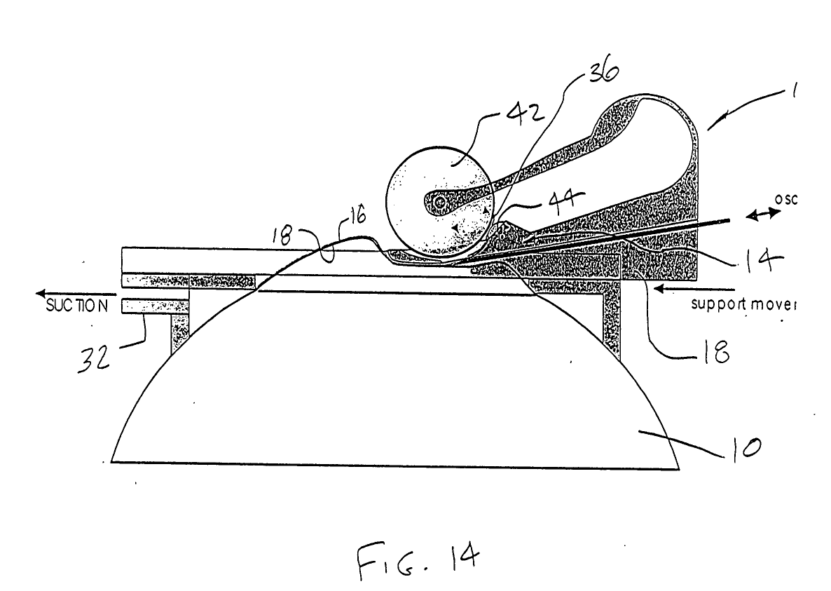

FIG. 9 is a diagram showing a top view of the eye 10 and the separator 14 located in a retracted position after the epithelial disk 34 as been formed. After the separator 14 is retracted, suction to the circumferential groove 32 is turned off and the epithelial separator device 12 is removed from the eye 10. Referring also to FIG. 10, after the epithelial separator device 12 is removed, a deepithelialized area 38 is exposed that corresponds to a shape and size of the area that the separator 14 contacted during travel.

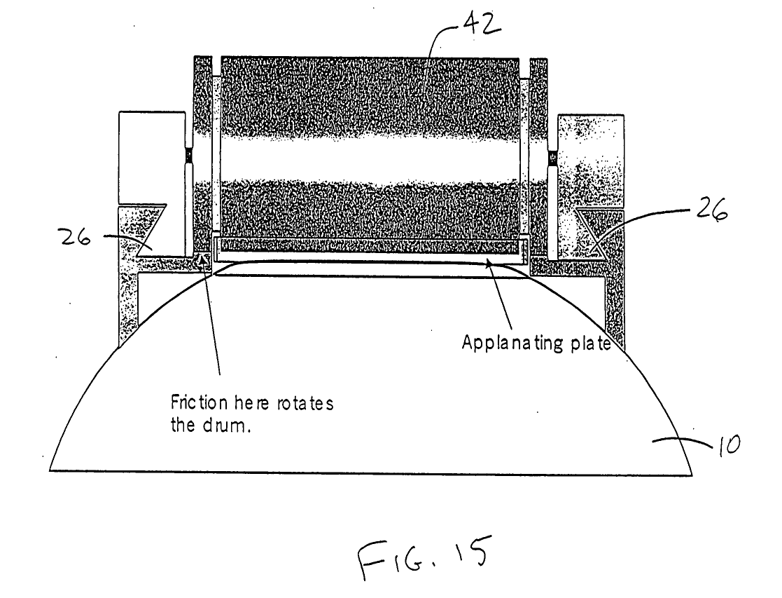

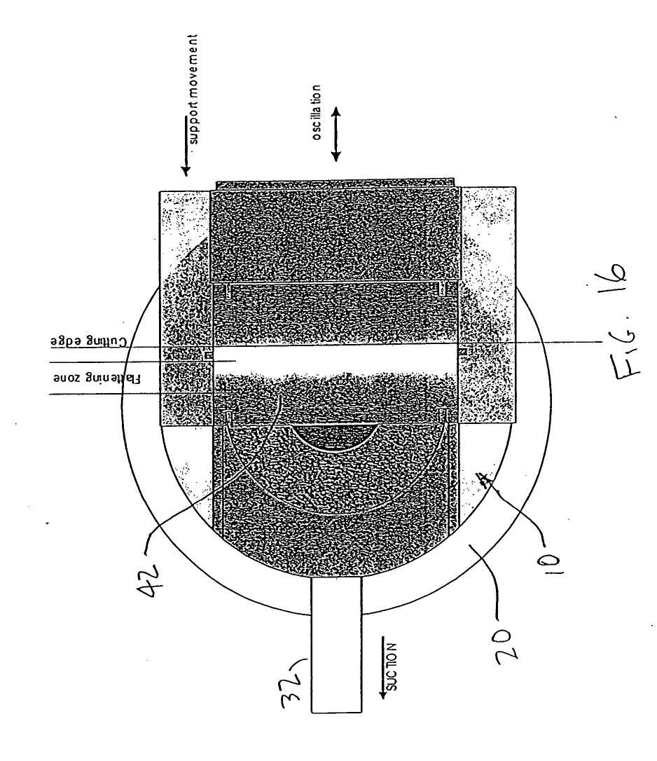

FIG. 11 shows a top view of the eye 10 after laser ablation is performed. The laser ablation forms an irradiated area 40 on the eye 10. Referring to FIG. 12, thereafter, the epithelium disk 34 is replaced on the corneal surface 18 of the eye 10 to aid in the healing process. Referring to FIG. 13, once replaced on the corneal surface 18, the epithelium disk 34 is preferably smoothly stretched into place.

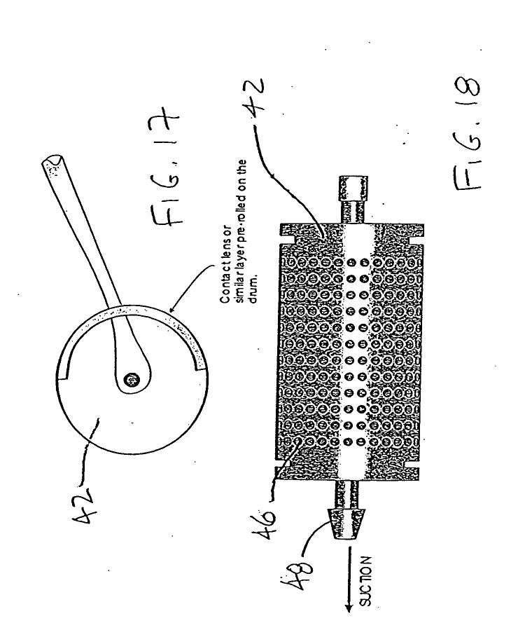

FIG. 14 is a diagram showing a side view of the eye 10 and the epithelial separator device 12 including rotating drum 42. To rotate the drum 42, the epithelial separator device 12 may include a rotating gear 44. The gear 44 could also be used to provide movement to the separator support 28. Referring also to FIGS. 15 and 16, front and top views, respectively, of the epithelial separator device 12, the rotating gears 44 could be bilaterally placed on the separator support 28. The oscillating device 30 can provide for rotation of the gears 44 and the gears 44 can travel on rails, for example toothed rails, which run parallel to the groove 26.

Since a typical thickness of an epithelial disk 36 includes about 50 microns, to preserve an epithelial disk 36, a separated epithelial disk 36 is rolled onto the drum 42. The drum 42 can include a diameter ranging from about 3 to about 9 mm and a length of about 12 mm. Referring also to FIG. 17, in one embodiment, to maintain integrity of the epithelial disk 36, the drum 42 can be coated with a hydrating and/or a conditioning substrate. The hydrating and/or conditioning substrate can include, for example, HEMA contact lenses, tissue culture media, silicone and biocompatible hydrogels. The hydrating and/or conditioning substrate can be removed from the drum after the epithelial disk 36 attaches on to the drum. Thereafter, the epithelial disk 36 can be removed from the drum 46 and replaced on the corneal surface 16, as described above.

FIG. 18 shows another embodiment of the drum 42 includes apertures 46 and a connector 48 that connects to a suction source (not shown). By applying suction to the apertures 46 of the drum 42, the epithelial disk 36 can be rolled onto the drum 42. Thereafter, the epithelial disk 36 can be removed from the drum 46 and replaced on the corneal surface 16, as described above.

FIG. 19 is a diagram representing a side view of the separator 14 removing the epithelial layer 16 from a Basal membrane 1900 of the eye 10. The epithelial layer 16 is made up of epithelial cells 1902. The epithelial layer 16 overlies the Basal membrane 1900. The Basal membrane 1900 is formed from a lamina densa 1904 of about 50 nm in thickness and an underlying lamina lucida 1906 of about 25 nm in thickness. The lamina densa 1906 overlies a Bowman's layer 1908. The epithelial layer 16 anchors to the Bowman's layer via a complex mesh of anchoring fibrils (type VII collagen) and anchoring plaques (type VI collagen) that interact with the lamina densa 1904 and the collagen fibrils of the Bowman's layer 1908. The Bowman's layer 1908 overlies a corneal stroma 1910.

The epithelial layer 16 is stratified, possessing 5 to 6 layers of epithelial cells 1902. The epithelial layer 16 is typically about 50 to 60 micrometers in thickness. Adjacent epithelial cells 1902 are held together by desmosomes 1912. The epithelial cells 1902 are held to the underlying basal membrane 1900 by hemidesmosomes 1914 and anchoring filaments. A bottom surface of the epithelial layer 16 includes numerous microvilli and microplicae, i.e., ridges, whose glycocalyx coat interacts with, and helps to stabilize, a precorneal tear film. New epithelial cells 1902 are derived from mitotic activity in the basal membrane 1900 layer. New epithelial cells 1902 displace existing cells both superficially and centripetally.

The separator 14 includes a blunt leading edge to push the epithelial cells 1902 as the separator 14 moves across the epithelial layer 16. The separator 14 has a thickness that is preferably between one cell layer thick and the thickness of the epithelial layer 16. More preferably, the separator has a thickness between two to three cell layers in thickness. The separator 14 preferably pushes the epithelial cells 1902 and does not exert a force that could disrupt the intercellular bonds such as the desmosomes 1912. The point of separating the epithelial layer 16 has been found to often occur at the border between the lamina densa 1904 and the lamina lucida 1906. The separator 14 preferably pushes the bottom two to three layers of epithelial cells 1902 which probably contain a majority of the shear strength of the epithelial layer 16.

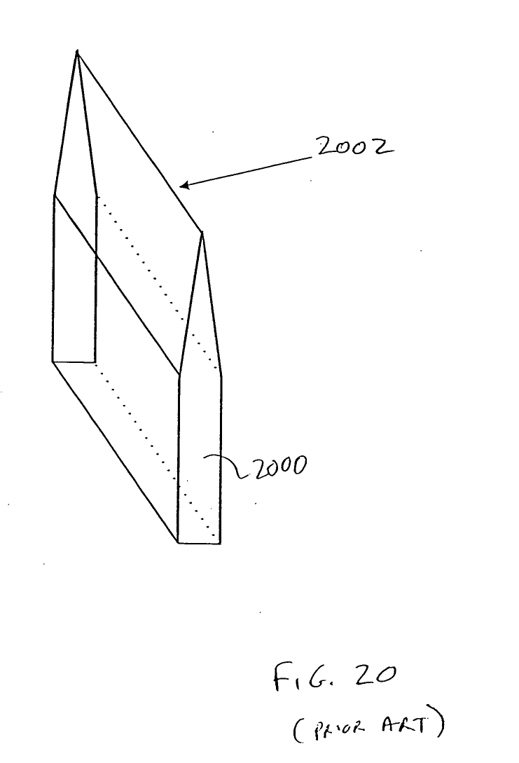

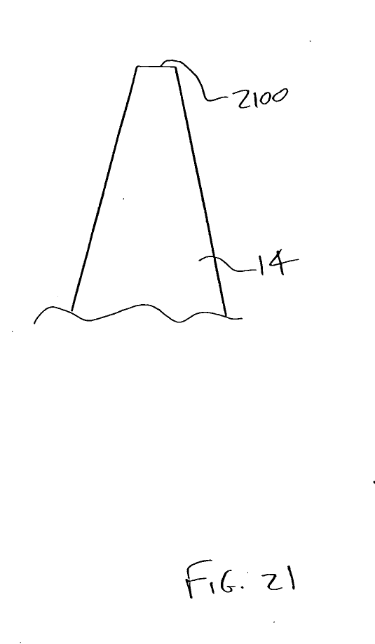

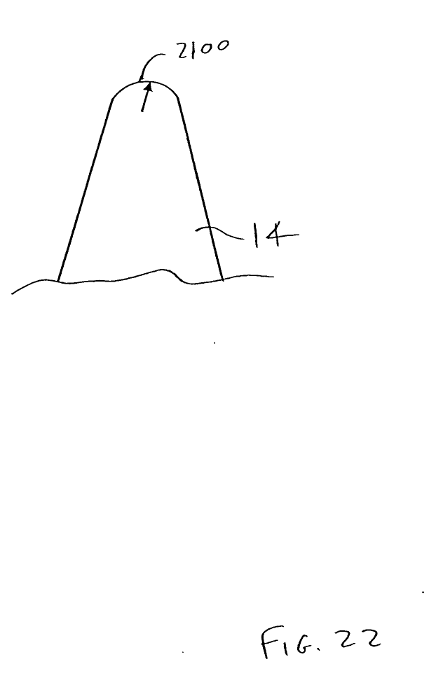

FIG. 20 is a diagram showing a perspective view of a known blade 2000. A leading edge 2002 of the blade 2000 is sharp and thus would not work well as a separator. The blade 2000 risks cutting the cornea.

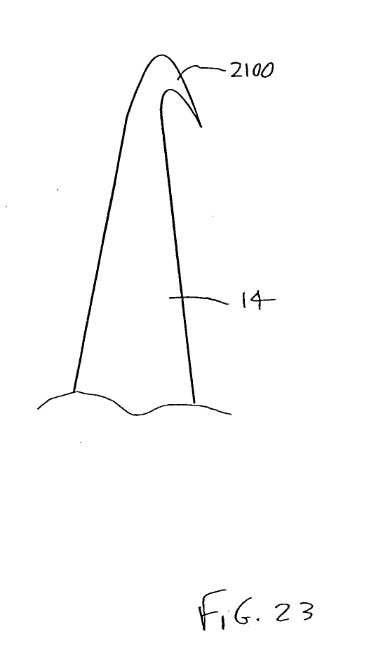

FIG. 21 is a diagram showing a side view of a leading edge 2100 of a separator 14 according to an embodiment. The leading edge 2100 of the separator 14 should not be too wide such that it will reduce the consistency with which the epithelial layer 16 is penetrated. The leading edge 2100 preferably includes a 5 to 25 micrometer width, and more preferably includes about a 15 micrometers width.

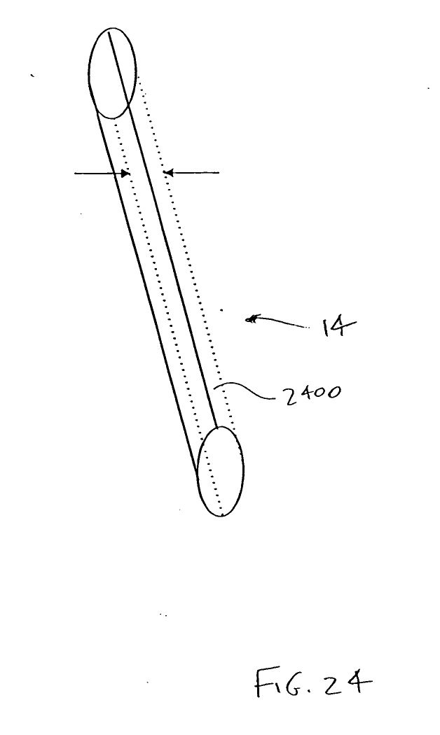

FIG. 22 is a diagram showing a side view of a separator's leading edge 2100 according to another embodiment. The leading edge 2100 is rounded instead of flat.

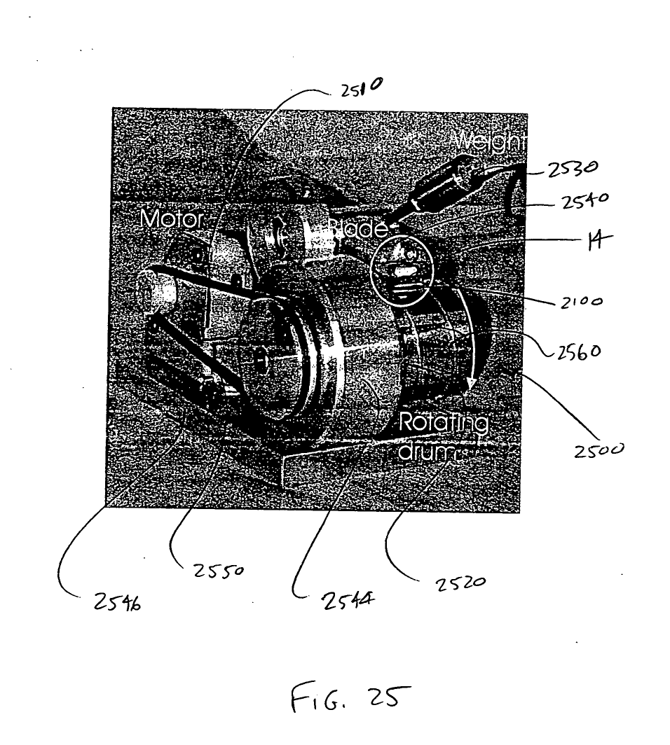

FIG. 23 is a diagram showing a side view of a separator's leading edge 2100 according to yet another embodiment. The separator 14 is constructed, for example, by bending the leading edge 2002 of the blade 2000 shown in FIG. 20. The leading edge 2001 preferably includes a diameter of about 5 to 25 micrometers, or a radius between about 2 to 13 micrometers, and more preferably includes a diameter of 15 micrometers.

FIG. 24 is a diagram showing a perspective view of a wire 2400 that could be used as the separator 14 according to a preferred embodiment. The wire 2400 includes a generally elliptical or circular cross-sectional shape. The wire 2400 includes a leading edge with a width of about 5 to 25 micrometers. The wire 2400 is preferably manufactured from a material that is strong enough to push the epithelium without breaking. Exemplary wire materials include titanium and its alloys, tungsten and its alloys, steel alloys and carbon fibers.

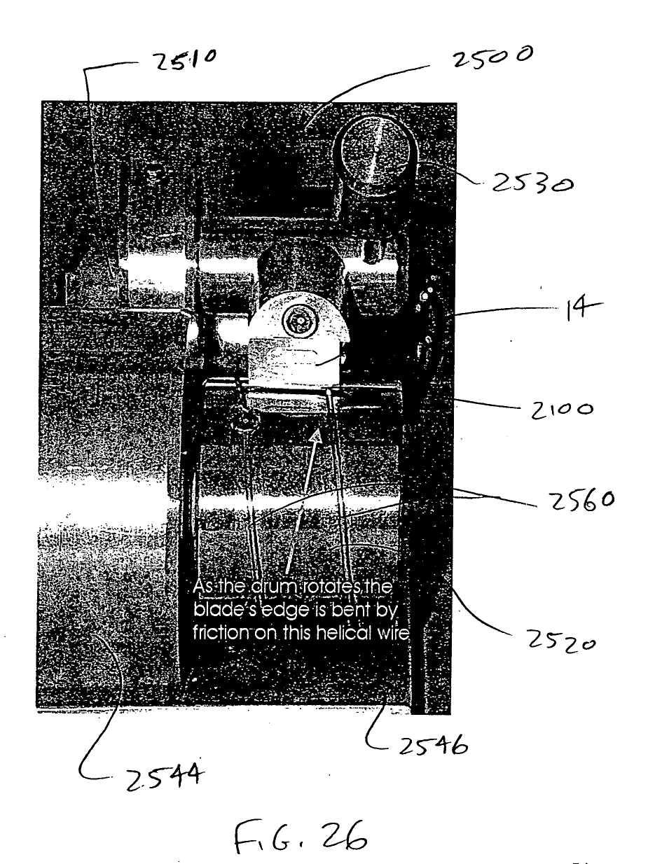

FIG. 25 shows a perspective view of an exemplary machine 2500 that is used to condition a separator 14 according to one embodiment. The machine 2500 conditions the separator 14 by changing a sharp edged separator to include a generally bent edge, for example, like the front edge of the separator 14 shown in FIG. 23.

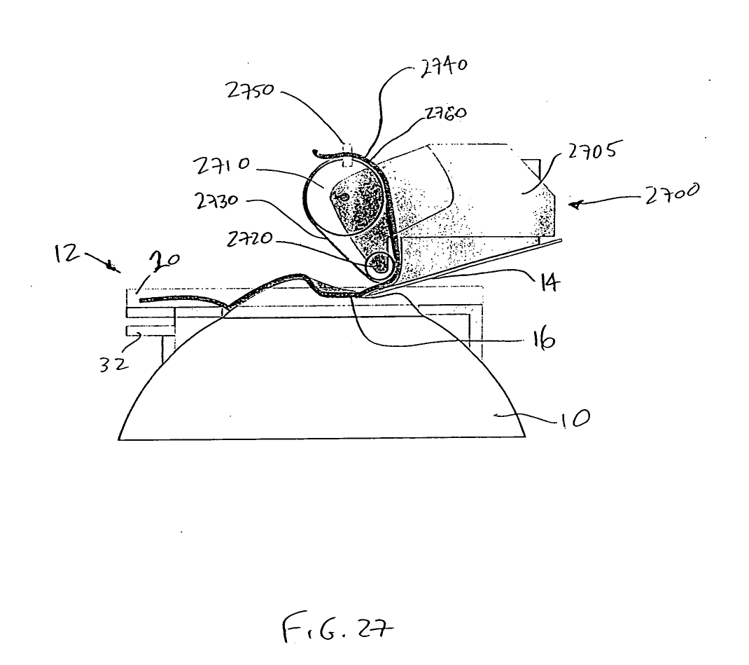

FIG. 26 shows a front view of the machine 2500 and separator 14. Referring to FIGS. 25 and 26, the machine 2500 includes a motor 2510, a rotating cylinder 2520, a weight 2530, or other way to hold the blade down, and a blade holder 2540. The motor 2510 and a housing 2544 of the cylinder 2520 rest on a platform 2546. The blade is held by, for example, a clamp. The blade's edge is substantially parallel to the axis of rotation of cylinder 2520. The blade's plane forms an angle between 0 and 20 degrees with the plane defined by the axis of the cylinder 2520 and the blade's edge. The motor 2510 connects to the cylinder 2520 via a belt 2550 to rotate the cylinder 2520. In another embodiment, the motor 2510 connects directly to the cylinder 2520 to rotate the cylinder.

The cylinder 2520 includes a helical wire 2560. The helical wire 2560 and the cylinder 2520 are manufactured from steel. This helical wire serves as a helical protrusion of the rotating drum. This helix has a pitch equal to the length of the blade's edge. The helix causes only one point of the blade to be conditioned at any given moment (the point of contact between the blade's edge and the helical wire). As the helical wire 2560 rotates along with drum 2520, the point of contact travels along the blade's edge, but the amount of conditioning is equal across the blade's length. The weight 2530, and the running time and rotations of the cylinder 2520 vary the shape and width of the leading edge 2100 of the separator 14. In one embodiment, a preferred separator 14 has been conditioned by asserting 20 mN of force on the separator 14 to the cylinder 2520 and operating the cylinder for about 45 second at 0.7 (seven-tenths) rotations/second.

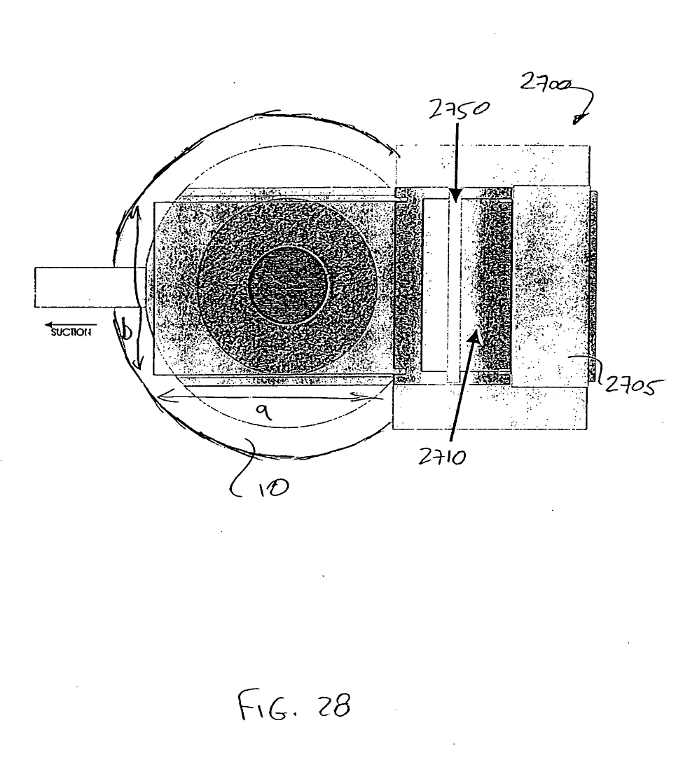

FIG. 27 shows a side view of one embodiment of a device 2700 for separating and preserving an epithelial layer 16. The device 2700 includes a body 2705, a first drum 2720 and a second drum 2730, and a belt 2730 connecting the first drum 2720 to the second drum 2730. The device 2700 accommodates a substrate, such as film 2740. Film 2740 is used to substantially preserve the epithelial layer 16 when the epithelial layer 16 is removed from the eye 10. The film 2740 can be held to the drum 2710 with a bar or clip 2750. Alternatively, the film 2740 can serve to connect the drums 2720 and 2730 and therefore eliminate the use of belt 2730.

FIG. 28 shows a top view of the device 2710 and how the device 2700 is used with the clip 2750. In one embodiment, the film 2740 is rolled on to the drum 2710 and under the clip 2750 (see also FIG. 27). The first drum 2710 turns as the second drum 2720 turns since they are connected by the belt 2730. The film 2740 lays on the belt 2730 and moves as the first drum 2710 and the second drum 2720 move. The film 2740 preferably removably adheres to the belt 2730 through cohesion.

The film 2740 includes an outer surface 2760. The outer surface 2760 is constructed to adhere to the epithelial layer 16 to provide mechanical stability to the epithelial layer 16 when the epithelial layer 16 is separated from the eye 10. The film 2740 includes a natural or synthetic polymer. An exemplary polymer includes HEMA (poly-2hydroxy-ethyl-methacrylate). The film 2740 includes a thickness from about 20 to about 100 micrometers. If the film 2740 is in the shape of a strip of film, a length (a) and a width (b) of the film 2740 is preferably longer and wider than the diameter of a separated epithelium layer 16.

The film 2740 is preferably hydrated to adhere the epithelial layer 16 to the film 2740. The level of hydration of the film 2740 controls adhesion to the film 2740. The hydrated film 2740 also helps to keep cracks from forming in the removed epithelial layer 16, and to help avoid the removed epithelial layer 16 from being torn or shrinking. In one embodiment, a surface of the epithelial layer 16 is dried, for example, with a sponge or with a compressed air flow. Thereafter, the film 2740 is placed on the epithelial layer 16. The epithelial layer 16 adheres to the film 2740 because of the difference in hydration levels between the epithelial layer and the film. Thereafter, the separator 14 is used to separate the epithelial layer 16. The film 2740 and the epithelial layer 16 are rolled onto the first and second drums 2710, 2720.

It should be appreciated that the strip of film 2740 does not have to be applied with the device 2700 and that the strip does not need to include a coating. Moreover, the film 2740 can be applied before or after removal of the epithelial layer 16, and can be manually applied instead of using the device 2700.

The film 2740 can include other shapes such as the shape of a disc. A way to attach the epithelial layer 16 to a disc, such as a contact lens, is to separate the epithelial layer 16 and remove the epithelial layer 16 to the side. The epithelial layer 16 is then smoothed with a sponge and dried with the sponge, compressed air or both. Thereafter, the removed epithelial layer 16 is placed on the film 2740. The epithelial layer 16 and the film 2740 are then dried, for example, with compressed air. After about 30 seconds of drying, the epithelial layer 16 is adhered to the film 2740 and can be more easily manipulated with a reduced risk of damage.

While the invention has been described above by reference to various embodiments, it will be understood that many changes and modifications can be made without departing from the scope of the invention. It is therefore intended that the foregoing detailed description be understood as an illustration of the presently preferred embodiments of the invention, and not as a definition of the invention. It is only the following claims, including all equivalents, which are intended to define the scope of this invention.

We claim:

1. A device for separating the epithelial layer of a cornea, the device comprising:
a separator having an edge, wherein the edge comprises a thickness thicker than the thickness of at least one epithelial cell and less thick than the thickness of the epithelial layer.

2. The device of claim 1 wherein the separator comprises a blade having a bent edge.

3. The device of claim 1 wherein the separator comprises a wire.

4. The device of claim 1 wherein the edge of the separator comprises approximately a trapezoid shape including a flat front edge.

5. The device of claim 4 wherein the flat front edge comprises a thickness of between about 5 and 25 micrometers.

6. The device of claim 1 wherein the edge of the separator comprises an approximately semi-cylindrical surface including a front edge.

7. The device of claim 6 wherein the semi-circular surface comprises a radius of between about 2 and 13 micrometers.

8. The device of claim 1 wherein the edge comprises a bent front edge to form a shape resembling a semicircle.

9. The device of claim 8 wherein the semicircular edge comprises m a diameter of between about 5 and 25 micrometers.

10. The device of claim 1 wherein the separator comprises an elliptical shaped wire.

11. The device of claim 10 wherein the edge that is used for m separating comprises a thickness from about 5 to about 25 micrometers.

12. The device of claim 1 wherein the separator comprises a circular shaped wire.

13. The device of claim 12 wherein the edge that is used for separating comprises a width from about 5 to about 25 micrometers.

14. A substrate for substantially preserving an epithelial layer of eye, wherein the substrate comprises:
a film having a first surface, wherein the first surface is constructed to adhere to the epithelial layer to provide mechanical stability to the epithelial layer that is separated from the eye.

15. The substrate of claim 14 wherein the film comprises a natural polymer.

16. The substrate of claim 14 wherein the film comprises a synthetic polymer.

17. The substrate of claim 14 wherein the film comprises a thickness from about 20 to about 100 micrometers.

18. The substrate of claim 14, wherein the film comprises the shape of a strip.

19. The substrate of claim 18, wherein the strip comprises a length and a width that is longer and wider than the diameter of a separated epithelium layer.

20. The substrate of claim 14 wherein the film comprises a HEMA material.

21. The substrate of claim 14 wherein the film comprises the shape of a disc.

22. The substrate of claim 21 wherein the disc comprises a HEMA material.

23. The substrate of claim 14 wherein a hydration level of the film controls adhesion to the film.

24. The substrate of claim 14 wherein the film is applied and adhered to the epithelium layer before the epithelium layer is removed from the eye.
